(19) United States
(12) Patent Application Publication
(10) Pub. No.: US 2008/0089861 A1
(43) Pub. Date: Apr. 17, 2008

(54) Combination therapy for treatment of demyelinating conditions

(76) Inventors: Gregory T. Went, Mill Valley, CA (US); Ivan Lieberburg, Berkeley, CA (US)

Correspondence Address: Adamas Pharmaceuticals, Inc., 1900 Powell Street, Suite 1050, Emeryville, CA, 94608, US

(21) Appl. No.: 11/827,251

(22) Filed: Jul. 10, 2007

Related U.S. Application Data
(60) Provisional application No. 60/819,955, filed on Jul. 10, 2006.

(51) Int. Cl. A61K 31/19; A61K 31/13; A61K 38/21; A61P 21/00; A61K 39/395; A61K 31/135

(52) U.S. Cl. 424/85.6; 424/141.1; 514/574; 514/656; 514/661

(57) ABSTRACT

The invention provides compositions and methods for treating autoimmune diseases such as multiple sclerosis. The compositions include a combination of an NMDA Receptor antagonist, such as memantine or rimantadine, and a fumarate agent, such as dimethyl fumarate.

### RELATED APPLICATIONS

This application claims priority from U.S. Provisional Application No. 60/819,955, filed Jul. 10, 2006. The contents of this applications are incorporated herein by reference in their entirety.

### FIELD OF THE INVENTION

This invention relates to compositions and methods for treating autoimmune diseases such as multiple sclerosis.

### BACKGROUND OF THE INVENTION

Myelin is a vital component of the central and peripheral nervous system. In this regard, the encapsulation of neuronal axons by the myelin sheath ensures that nerve signals are efficiently transmitted between the brain and peripheral nerves. Serious neurological disorders arise with devastating consequences to patients' lives if the integrity of the myelin sheath is compromised. Exemplary diseases characterized by excessive demyelination include multiple sclerosis (MS), disseminated necrotizing leukoencephalopathy (DNL), and progressive multifocal leukoencephalopathy (PML).

### SUMMARY OF THE INVENTION

In one aspect, the invention provides a method for treating multiple sclerosis or other demyelinating condition by administering one or more amino-adamantane-derived NMDA receptor antagonists, such as memantine, rimantadine, and amantadine in combination with a fumarate agent, such as dimethyl fumarate.

In some embodiments, the combination is additionally administered along with one or more multiple sclerosis agents or treatments, such as interferon-βs (e.g., BETASERON™ REBIF™, or AVONEX™), or other MS agents, such COPAXONE™, ANTEGREN™, NOVANTRONE™, or ZENEPAX™. In this embodiment, the combination and additional multiple sclerosis agent are administered to a subject having multiple sclerosis or other demyelinating condition, such that the multiple sclerosis or other demyelinating condition is treated or at least partially alleviated. The combination and further multiple sclerosis agent may be administered as part of a pharmaceutical composition, or as part of a combination therapy. In another embodiment, a patient is diagnosed, e.g., to determine if treatment is necessary, whereupon a combination therapy in accordance with the invention is administered to treat the patient.

Demyelinating conditions include, for example, multiple sclerosis (MS); progressive multifocal leukoencephalopathy (PML); disseminated necrotizing leukoencephalopathy (DNL); acute disseminated encephalomyelitis; Schilder disease, central pontine myelinolysis (CPM); radiation necrosis; and Binswanger disease (SAE); Guillain-Barre Syndrome; leukodystrophy; acute disseminated encephalomyelitis (ADEM); acute transverse myelitis; acute viral encephalitis; adrenoleukodystrophy (ALD); adrenomyeloneuropathy; AIDS-vacuolar myelopathy; experimental autoimmune encephalomyelitis (EAE); experimental autoimmune neuritis (EAN); HTLV-associated myelopathy; Leber's hereditary optic atrophy; subacute sclerosing panencephalitis; and tropical spastic paraparesis.

Also provided for compositions that include amino-adamantane-derived NMDA receptor antagonist agents in combination with the fumarate agent, and are used in the treatment of patients suffering from MS or other demyelinating condition.

The NMDA receptor antagonist agents and fumarate agent may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of NMDA receptor antagonist agent and a multiple sclerosis agent is typically effective to reduce symptoms and to enable an observation of a reduction in symptoms.

The amino-adamantane-derived NMDA receptor antagonist agent can be, e.g., an uncompetitive NMDA receptor antagonist. The NMDA receptor antagonist can be, e.g., (1-amino-3,5-dimethyladamantane), rimantadine (1-(1-aminoethyl)adamantane), or amantadine (1-amino-adamantane), pharmaceutically acceptable salts, and combinations thereof. Other amino-adamantane-derived NMDA receptor antagonist agents are those described in U.S. Pat. No. 5,061,703. Generally, for memantine the dosage is from about 5 to about 20 mg/day, for amantadine the dosage is from about 50 to about 200 mg/day, and for rimantadine the dosage is from about 50 to about 200 mg/day.

The fumarate agent can be any fumaric acid derivative that, when administered with an NMDA receptor antagonist to a subject, ameliorates the symptoms of an auto-immune or CNS-related condition in the subject. The fumarate agent preferably has minimal side effects when administered as part of the combination. Examples of fumarate agents include a fumaric acid ester. Preferably, it is efficacious when administered orally. An example of a suitable orally delivered fumaric acid ester is dimethyl fumarate

The combination of the NMDR receptor antagonist and fumarate agent may also be adiministered with one or more additional multiple sclerosis agents. These include, e.g., β-interferons, glatiramer acetate, natalizumab, mitoxanthrone and daclizumab. When interferon-β1a (i.e., AVONEX™) is used, it is administered at a dosage of about 7.5 to about 30 μg preferably intramuscularly, once a week. When interferon-β1a (i.e., REBIF™) is used, it is administered at a dosage of about 11 μg to about 44 μg™, preferably subcutaneously and preferably three times a week. When interferon-β1b (e.g., BETASERON™) is used, it is administered at a dosage of about 50 μg to about 250 μg, preferably subcutaneously and preferably every other day. When glatiramer acetate (e.g., COPAXONE™) is used, it is administered at a dosage of about 5 mg to about 20 mg, e.g., 10, 15 or 20 mg, and is preferably administered subcutaneously, preferably daily. When natalizumab (ANTEGREN™) is used, it is administered at a dosage of about 1.5 mg/kg to 6 mg/kg by intravenous infusion, preferably once every four weeks. When mitoxanthrone (NOVANTRONE™) is used for reducing neurologic disability and/or the frequency of clinical relapses in patients with secondary (chronic) progressive, progressive relapsing, or worsening relapsing remitting multiple sclerosis, the recommended dosage of NOVANTRONE is about 3 to about 12 mg/m² given as a short (approximately 5 to 15 minutes) intravenous infusion every 3 months. When daclizumab (ZENAPAX™) is used, it is administered at a dosage of about 0.25 to about 1 mg/kg (intravenous) every 14 days, for a total of 5 doses.

In one embodiment, a combination therapy for MS or other demyelinating condition includes memantine and a fumarate agent, such as dimethyl fumarate, and, optionally, β-interferon, for treating a patient in need of such treatment. In another embodiment, a combination therapy for includes rimantadine and a fumarate agent, such as dimethyl fumarate, and, optionally β-interferon, for treating a patient in need of such treatment. In an embodiment, a combination therapy for MS or other demyelinating condition includes amantadine and a fumarate agent, such as dimethyl fumarate, and, optionally, β-interferon, for treating a patient in need of such treatment.

In another embodiment, a combination therapy for MS or other demyelinating condition includes memantine and a fumarate agent, such as dimethyl fumarate, and, optionally, glatiramer, for treating a patient in need of such treatment. In one embodiment, a combination therapy for MS or other demyelinating condition includes rimantadine and a fumarate agent, such as dimethyl fumarate, and, optionally, glatiramer, for treating a patient in need of such treatment. In an embodiment, a combination therapy for MS or other demyelinating condition includes amantadine and a fumarate agent, such as dimethyl fumarate, and, optionally, glatiramer, for treating a patient in need of such treatment.

In yet another embodiment, a combination therapy for MS or other demyelinating condition includes memantine and natalizumab, for treating a patient in need of such treatment. In one embodiment, a combination therapy for MS or other demyelinating condition includes rimantadine and natalizumab, for treating a patient in need of such treatment. In yet another embodiment, a combination therapy for MS or other demyelinating condition includes amantadine and natalizumab, for treating a patient in need of such treatment.

In yet another embodiment, a combination therapy for MS or other demyelinating condition includes memantine and daclizumab, for treating a patient in need of such treatment. In one embodiment, a combination therapy for MS or other demyelinating condition includes rimantadine and daclizumab, for treating a patient in need of such treatment. In yet another embodiment, a combination therapy for MS or other demyelinating condition includes amantadine and daclizumab, for treating a patient in need of such treatment. In yet another embodiment, a combination therapy for MS or other demyelinating condition includes memantine and mitoxanthrone, for treating a patient in need of such treatment. In one embodiment, a combination therapy for MS or other demyelinating condition includes rimantadine and mitoxanthrone, for treating a patient in need of such treatment. In yet another embodiment, a combination therapy for MS or other demyelinating condition includes amantadine and mitoxanthrone, for treating a patient in need of such treatment.

Administration of the combination (both or individually) may be administered in any suitable route, e.g, subcutaneously, intramuscularly, intravenously, orally, topically, or intranasally.

Also provided are kits for treating patients having multiple sclerosis or other demyelinating condition, comprising a therapeutically effective dose of an agent for treating or at least partially alleviating the symptoms of multiple sclerosis or other demyelinating condition (e.g., β-interferons, glatiramer acetate, natalizumab, or daclizumab), and an NMDA receptor antagonist, either in the same or separate packaging, and instructions for its use.

Pharmaceutical compositions comprising an NMDA receptor antagonist and a multiple sclerosis agent, in an effective amount(s) to treat multiple sclerosis or other demyelinating condition, are also included in the invention.

In one aspect, the pharmaceutical composition comprises an NMDA receptor antagonist; a second agent, wherein the agent is a fumarate agent; and a pharmaceutically acceptable carrier. In some embodiments, the fumarate agent is a fumaric acid ester. In some embodiments, the fumaric acid ester is a dialkyl fumarate. Preferably, the fumarate agent is efficacious, safe and tolerated when administered orally to a subject.

In some embodiments, at least one of the NMDA receptor antagonist or the second agent is provided in an extended release dosage form.

In some embodiments, the NMDA receptor antagonist has a dC/dT less than about 80% of the rate for the IR formulation.

In some embodiments, the NMDA receptor antagonist has a Cₘₐₓ/Cₘₑₐₙ of approximately 1.6 or less, approximately 2 hours to at least 12 hours after the NMDA receptor antagonist is introduced into a subject.

In some embodiments, the relative Cratio.var of the NMDA receptor antagonist and the second fumarate agaent is less than 100% from 2 hour to 12 hours post administration.

In some embodiments, the relative Cratio.var of the NMDA receptor antagonist and the fumarate agent is less than 70% of the corresponding IR formulation from 2 hour to 12 hours post administration.

In some embodiments, the second agent is dimethyl fumarate.

In some embodiments, the NMDA receptor antagonist is memantine.

In some embodiments, the pharmaceutical composition is formulated for oral, transnasal, parenteral, , subtopical transepithelial, transdermal patch, subdermal, or inhalation delivery. In some embodiments, the pharmaceutical composition is formulated for oral delivery.

In some embodiments, the pharmaceutical composition is formulated as a suspension, capsule, tablet, suppository, lotion, or patch.

In some embodiments, the NMDA receptor antagonist is memantine and the second agent is dimethyl fumar

Also provided by the invention is a method of treating a demyelinating condition comprising administering to a subject having a demyelinating condition a combination therapy including an NMDA receptor antagonist and a fumarate agent, such that the demyelinating condition is treated or at least partially alleviated.

In some embodiments, the amount of the NMDA receptor antagonist and/or the a multiple sclerosis agent is effective to reduce symptoms and to enable an observation of a reduction in symptoms.

In some embodiments, the demyelinating condition is selected from the group consisting of multiple sclerosis (MS); progressive multifocal leukoencephalopathy (PML); disseminated necrotizing leukoencephalopathy (DNL); acute disseminated encephalomyelitis; Schilder disease, central pontine myelinolysis (CPM); radiation necrosis; Binswanger disease (SAE); Guillain-Barre Syndrome; leukodystrophy; acute disseminated encephalomyelitis (ADEM); acute transverse myelitis; acute viral encephalitis; adrenoleukodystrophy (ALD); adrenomyeloneuropathy; AIDS-vacuolar myelopathy; experimental autoimmune encephalomyelitis (EAE); experimental autoimmune neuritis (EAN); HTLV-associated myelopathy; Leber's hereditary optic atrophy; subacute sclerosing panencephalitis; and tropical spastic paraparesis.

In some embodiments, the demyelinating condition is multiple sclerosis.

In some embodiments, the NMDA receptor antagonist is selected from the group consisting of memantine, rimantadine, and amantadine.

In some embodiments, the fumarate agent is a fumarate ester.

In some embodiments, the fumarate agent is a dialkyl fumarate.

In some embodiments, the fumarate agent is administered orally to the subject.

In some embodiments, the fumarate agent is dimethyl fumarate.

In some embodiments, the subject is a human.

In some embodiments, the method further comprises administering an additional multiple sclerosis agent to the subject.

In some embodiments, the multiple sclerosis agent is selected from the group consisting of β-interferons, glatiramer acetate, natalizumab, mitoxanthrone and daclizumab.

In some embodiments, the multiple sclerosis agent is an interferon-β.

In some embodiments, the β-interferon is interferon-β1a, interferon-β1b, or interferon-β2.

In some embodiments, the β-interferon is interferon-β1a, (AVONEX™) administered at a dosage of from about 7.5 μg to about 30 μg per week.

In some embodiments, the interferon-β1b is administered intramuscularly.

In some embodiments, the β-interferon is interferon-β1a (REBIF™) administered at a dosage of about 11 to about 44 μg three times a week.

In some embodiments, the β-interferon is interferon-β1b (BETASERON™) administered at a dosage of from about 50 μg to about 250 μg three times a week.

In some embodiments, the interferon-β1b is administered subcutaneously.

In some embodiments, the multiple sclerosis agent is glatiramer acetate (COPAXONE™) administered at a dosage of from about 5 mg to about 20 mg per day.

In some embodiments, the glatiramer acetate is administered subcutaneously.

In some embodiments, the multiple sclerosis agent is mitoxanthrone (NOVANTRONE™) administered at a dose of from about 3 mg/m² to about 12 mg/m² for about 5 to 15 minutes intravenously.

In some embodiments, the dose is given about every 3 months.

In some embodiments, the treatment is administered orally.

In some embodiments, the amount of the NMDA receptor antagonist is at least about 5 to 200 mg per day.

In some embodiments, the dose of NMDA receptor antagonist is at least about 50 to 200 mg per day.

In some embodiments, the dose of NMDA receptor antagonist is at least about 50 to 100 mg per day.

In some embodiments, the dose of NMDA receptor antagonist is at least about 5 to 20 mg per day.

In some embodiments, the dose of the fumarate agent is about 1 to 1000 mg per day.

In some embodiments, the dose of the fumarate agent is about 125 to 875 mg per day.

In some embodiments, the dose of the fumarate agent is about 250 to 750 mg per day.

In some embodiments, the dose of the fumarate agent is about 375 to 625 mg per day.

In some embodiments, the dose of the fumarate agent is about 120, 260 or 720 mg per day.

In another aspect the invention provides a method of treating a demyelinating condition, comprising administering to a patient in need thereof a pharmaceutical composition comprising an NMDA receptor channel antagonist and a fumarate agent in an amount effective to treat the demyelinating condition in the patient.

In a further aspect the invention provides a method of treating a demyelinating condition, comprising diagnosing a patient in need of treatment and administering to a patient in need thereof a combination therapy including an NMDA receptor channel antagonist and a fumarate agent such that the demyelinating condition is treated or at least partially alleviated.

Also provided by the invention is a kit for treating a patient having multiple sclerosis, comprising a therapeutically effective dose of a fumarate agent and a NMDA receptor antagonist, either in the same or separate packaging, and instructions for its use.

In some embodiment, the kit includes an additional agent for treating multiple sclerosis. In some embodiments, the additional agent is selected from the group consisting of β-interferons, glatiramer acetate, natalizumab, mitoxanthrone and daclizumab.

In some embodiments, the NMDA receptor antagonist is selected from the group consisting of memantine, rimantadine, and amantadine.

In some embodiments, the fumarate agent is dimethyl fumarate.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting. All parts and percentages are by weight unless otherwise specified.

### DETAILED DESCRIPTION OF THE INVENTION

The present invention provides methods and compositions for treating or preventing The invention provides methods and compositions for treating or preventing demyelinating disorders (e.g., multiple sclerosis) and involves administering to a mammal in need thereof a combination that includes an NMDA receptor antagonist and a fumarate derivative, referred to herein as a fumarate agent. Desirably, either of these two agents, or even both agents, is formulated for extended release, thereby providing a concentration over a desired time period that is high enough to be therapeutically effective but low enough to avoid adverse events associated with excessive levels of either component in the subject. The combination is administered such that symptoms associated with the demyelinating disease being treated are alleviated or prevented, or alternatively, such that the progression of the demyelinating disease is reduced.

This combination is administered such that symptoms associated with the autoimmune, e.g., demylienating condition, being treated are alleviated or prevented, or alternatively, such that progression of the condition is reduced. Desirably, either of these two agents, or even both agents, is formulated for extended release, thereby providing a concentration and optimal concentration ratio over a desired time period that is high enough to be therapeutically effective but low enough to avoid adverse events associated with excessive levels of either component in the subject.

The combination may additionally be administered with an additional demyelinating disease antagonist.

NMDA Receptor Antagonist

Any NMDA receptor antagonist can be used in the methods and compositions of the invention, particularly those that are non-toxic when used in the combination of the invention. The term “nontoxic” is used in a relative sense and is intended to designate any substance that has been approved by the United States Food and Drug Administration (“FDA”) for administration to humans or, in keeping with established regulatory criteria and practice, is susceptible to approval by the FDA or similar regulatory agency for any country for administration to humans or animals.

The NMDA receptor antagonist may be an amino-adamantane compound including, for example, memantine (1-amino-3,5-dimethyladamantane), rimantadine (1-(1-aminoethyl)adamantane), amantadine (1-amino-adamantane), as well as pharmaceutically acceptable salts thereof. Memantine is described, for example, in U.S. Pat. Nos. 3,391,142, 5,891,885, 5,919,826, and 6,187,338. Amantadine is described, for example, in U.S. Pat. No. 3,152,180, 5,891,885, 5,919,826, and 6,187,338. Additional aminoadamantane compounds are described, for example, in U.S. Pat. Nos. 4,346,112, 5,061,703, 5,334,618, 6,444,702, 6,620,845, and 6,662,845. All of these patents are hereby incorporated by reference.

Further NMDA receptor antagonists that may be used include, for example, ketamine, eliprodil, ifenprodil, dizocilpine, neramexane, remacemide, iamotrigine, riluzole, aptiganel, phencyclidine, flupirtine, celfotel, felbamate, spermine, spermidine, levemopamil, dextromethorphan ((+)-3-hydroxy-N-methylmorphinan) and its metabolite, dextrorphan ((+)-3-hydroxy-N-methylmorphinan), a pharmaceutically acceptable salt or ester thereof, or a metabolic precursor of any of the foregoing.

The NMDA receptor antagonist may be provided so that it is released at a dC/dT that is significantly reduced over an immediate release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5 or at least 0.8. In addition, the NMDA receptor antagonist may be provided such that it is released at rate resulting in a Cₘₐₓ/Cₘₑₐₙ of approximately 2 or less for approximately 2 hours to at least 8 hours after the NMDA receptor antagonist is introduced into a subject. The pharmaceutical composition may be formulated to provide memantine in an amount ranging between 1 and 80 mg/day, 5 and 40 mg/day, or 10 and 20 mg/day; amantadine in an amount ranging between 25 and 500 mg/day, 25 and 300 mg/day, or 100 and 300 mg/day; dextromethorphan in an amount ranging between 1-5000 mg/day, 1-1000 mg/day, and 100-800 mg/day, or 200-500 mg/day. Pediatric doses will typically be lower than those determined for adults.

Table 1 shows exemplary the pharmacokinetic properties (e.g., Tmax and T½) of memantine, amantadine, and rimantadine.

TABLE 1
Pharmacokinetics and Tox in humans for selected NMDAr antagonists

| | Human | | | |
|---|---|---|---|---|
| | PK (t½) | Tmax in | Normal | Dose Dependent |
| Compound | in hrs | hrs | Dose | Tox |
| Memantine | 60 | 3 | 10-20 mg/day, | Dose escalation |
| | | | starting at 5 mg | required, |
| | | | | hallucination |
| Amantadine | 15 | 3 | 100-300 mg/day | Hallucination |
| Rimantadine | 25 | 6 | 100-200 mg/day | Insomnia |

Fumarate Agent

The fumarate agent can be any fumaric acid derivative that, when administered with an NMDA receptor antagonist to a subject, ameliorates the symptoms of an auto-immune or CNS-related condition in the subject. The fumarate agent preferably has minimal side effects when administered as part of the combination.

Examples of fumarate agents include a fumaric acid ester. Preferably, it is efficacious when administered orally. An example of a suitable orally delivered fumaric acid ester is dimethyl fumarate, which is also known as BG-12. The fumarate agent can include one or more fumaric acid derivatives, e.g., a dialkyl fumarate alone, a monoalkyl ester salt thereof, or a combination of both. An example of a dialkyl fumarate is dimethyl fumarate. Fumarate agents are disclosed in e.g., U.S. Pat. Nos. 6,355,676; 6,359,003, and 6,436,992 Fumaric acid derivatives, including alkyl hydrogen fumarates, are disclosed in DE 197 21 099.6 and DE 198 53 487.6. Alkyl hydrogen fumarates and dialkyl fumarates are disclosed in DE 198 53 487.6 and DE 198 39 566.3.

In some embodiments, the fumarate agent includes a compound from the group consisting of calcium, magnesium, zinc and iron salts of fumaric acid monoalkyl esters of the general formula [(C₁-C₅-Alkyl-OOC)HCCH(COO]ₐA, optionally in admixture with dialkyl fumarate of the formula (C₁-C₅-Alkyl-OOC)HCCH(COO-C₁-C₅-Alkyl)], wherein A is a bivalent cation from the series consisting of Ca, Mg, Zn or Fe or a monovalent cation from the series consisting of potassium or sodium, respectively, and n denotes the numeral 1 or 2 depending on the type of cation, optionally together with commonly used pharmaceutical excipients.

The fumarate agent may additionally include a compounds of alkyl hydrogen fumaric acid of the general formula (ROOC)HCCHCOOH, optionally in admixture with dialkyl fumarate of the formula (R₁OOC)HCCH(COOR₂), wherein R, R₁, R₂ maybe the same or different and each of R, R₁, R₂ is an alkyl group having 1 to 5 carbon atoms (C₁-C₅ alkyl); and, optionally, commonly used pharmaceutical excipients and carriers.

In some embodiments, the fumarate agent contains the calcium salt of the fumaric acid monomethyl ester, the calcium salt of the fumaric acid monomethyl ester in admixture with dimethyl fumarate or the relevant salts of the fumaric acid monoethyl ester.

The fumarate agent can include preparations containing the calcium salt of the fumaric acid monoalkyl ester or the fumaric acid alkyl ester in the form of the free acid in an amount of 10 to 300 mg, the total weight of the active ingredients being in some embodiments 10 to 300 mg.

In some embodiments, the fumarate agent contains 10 to 290 parts by weight of the calcium salt of the fumaric acid monoalkyl ester and 290 to 10 parts by weight of dimethyl fumarate as well as 1 to 50 parts by weight of the zinc salt of the fumaric acid monoalkyl ester or 1 to 250 parts by weight of the calcium salt of the fumaric acid monoalkyl ester, 250 to 10 parts by weight of dimethyl fumarate, 1 to 50 parts by weight of the magnesium salt of the fumaric acid monoalkyl ester and 1 to 50 parts by weight of the zinc salt of the fumaric acid monoalkyl ester or the monomethyl ester, respectively, the total weight of the active ingredients being 30 to 300 mg. The fumarate agent may optionally include methyl hydrogen fumarate in an amount of 10 to 300 mg.

In some embodiments, the fumarate agent, e.g., dimethly fumarate, is administered in a range of about 1-1000 mg/day for an adult subject. In some embodiments, the dose is 100-750 mg/day, 200-600 mg/day, 250-550 mg/day administered 1-6 times per day. Examples of doses are 120 mg, 360 mg, or 720 mg administered 1-6 times per day.

In addition to the specific combinations disclosed herein, combinations made of an NMDA receptor antagonist such as an aminoadamantane compound and a fumarate agent may be identified by testing the ability of a test combination to lessen the symptoms associated with an auto-immune disease such as multiple sclerosis.

For a specified range a physician or other appropriate health professional will typically determine the best dosage for a given patient, according to his sex, age, weight, pathological state and other parameters. In some cases, it may be necessary to use dosages outside of the ranges stated in pharmaceutical packaging insert to treat a subject. Those cases will be apparent to the prescribing physician or veterinarian.

In some embodiments, the combinations of the invention achieve therapeutic levels while minimizing debilitating side-effects that are usually associated with immediate release formulations. Furthermore, as a result of the delay in the time to obtain peak plasma level and the potentially extended period of time at the therapeutically effective plasma level, the dosage frequency may be reduced to, for example, once or twice daily dosage, thereby improving patient compliance and adherence.

Accordingly, the combination of the invention allows the NMDA receptor antagonist and the fumarate to be administered in a combination that improves efficacy and avoids undesirable side effects of both drugs. For example, side effects including psychosis and cognitive deficits associated with the administration of NMDA receptor antagonists may be lessened in severity and frequency through the use of controlled-release methods that shift the Tmax to longer times, thereby reducing the dC/dT of the drug. Reducing the dC/dT of the drug not only increases Tmax, but also reduces the drug concentration at Tmax and reduces the Cmax/Cmean ratio providing a more constant amount of drug to the subject being treated over a given period of time and reducing adverse events associated with dosing. Similarly, side effects associated with the use of fumarate agents may be reduced in severity and frequency through controlled release methods as well.

In certain embodiments, the combinations provide additive effects. Additivity is achieved by combining the active agents without requiring controlled release technologies. In other embodiments, particularly when the pharmacokinetic profiles of the combined active pharmaceutical ingredients are dissimilar, controlled release formulations optimize the pharmacokinetics of the active pharmaceutical agents to reduce the variability of the Cratio over time. Reduction of Cratio variability over a defined time period enables a concerted effect for the agents over that time, maximizing the effectiveness of the combination. The Cratio variability (“Cratio.var”) is defined as the standard deviation of a series of Cratios taken over a given period of time divided by the mean of those Cratios multiplied by 100%. The Cratio for the controlled release formulation is more consistent than for the IR administration of the same drug combination over any significant time period, including shortly after administration and at steady state. This is evidenced by a lower Cratio.var for the controlled release formulation of the present invention relative to an IR administration.

Modes of Administration

The combination of the invention may be administered in either a local or systemic manner or in a depot or sustained release fashion. In one embodiment, the NMDA receptor antagonist, the fumarate, or both agents are formulated oral delivery.

In one embodiment, one or both components are formulated to provide fore controlled, extended release (as described herein). For example, a pharmaceutical composition that provides controlled release of the NMDA receptor antagonist, the fumarate agent, or both may be prepared by combining the desired agent or agents with one or more additional ingredients that, when administered to a subject, causes the respective agent or agents to be released at a targeted rate for a specified period of time. These agents may be delivered preferably in an oral, transdermal or intranasal form.

The two components are preferably administered in a manner that provides the desired effect from the first and second components in the combination. Optionally, the first and second agents are admixed into a single formulation before they are introduced into a subject. The combination may be conveniently sub-divided in unit doses containing appropriate quantities of the first and second agents. The unit dosage form may be, for example, a capsule or tablet itself or it can be an appropriate number of such compositions in package form. The quantity of the active ingredients in the unit dosage forms may be varied or adjusted according to the particular need of the condition being treated.

Alternatively, the NMDA receptor antagonist and the fumarate agent of the combination are not be mixed until after they are introduced into the subject. Thus, the term “combination” encompasses embodiments where the NMDA receptor antagonist and the fumarate agent are provided in separate formulations and are administered sequentially. For example, the NMDA receptor antagonist and the fumarate agaent are administered to the subject separately within 2 days, 1 day, 18 hours, 12 hours, one hour, a half hour, 15 minutes, or less of each other. Each agent may be provided in multiple, single capsules or tablets that are administered separately to the subject. Alternatively, the NMDA receptor antagonist and the fumarate agent are separated from each other in a pharmaceutical composition such that they are not mixed until after the pharmaceutical composition has been introduced into the subject. The mixing may occur just prior to administration to the subject or well in advance of administering the combination to the subject.

If desired, the NMDA receptor antagonist and the fumarate agent are administered to the subject in association with other therapeutic modalities, e.g., drug, surgical, or other interventional treatment regimens. Where the combination includes a non-drug treatment, the non-drug treatment may be conducted at any suitable time so long as a beneficial effect from the co-action of the combination and the other therapeutic modalities is achieved. For example, in appropriate cases, the beneficial effect is still achieved when the non-drug treatment is temporally removed from the administration of the therapeutic agents, perhaps by days or even weeks.

Formulations for Specific Routes of Administration

Combinations can be provided as pharmaceutical compositions that are optimized for particular types of delivery. For example, pharmaceutical compositions for oral delivery are formulated using pharmaceutically acceptable carriers that are well known in the art. The carriers enable the agents in the combination to be formulated, for example, as a tablet, pill, capsule, solution, suspension, sustained release formulation; powder, liquid or gel for oral ingestion by the subject.

Alternatively, the compositions of the present invention may be administered transdermally via a number of strategies, including those described in U.S. Pat. Nos. 5,186,938, 6,183,770,4,861,800 and WO 89/09051. Providing the drugs of the combination in the form of patches is particularly useful given that these agents have relatively high skin fluxes.

Pharmaceutical compositions containing the NMDA receptor antagonist and/or fumarate agent may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

In some embodiments, for example, the composition may be delivered intranasally to the cribriform plate rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The combination may optionally be formulated for delivery in a vessel that provides for continuous long-term delivery, e.g., for delivery up to 30 days, 60 days, 90 days, 180 days, or one year. For example the vessel can be provided in a biocompatible material such as titanium. Long-term delivery formulations are particularly useful in subjects with chronic conditions, for assuring improved patient compliance, and for enhancing the stability of the combinations. Formulations for continuous long-term delivery are provided in, e.g., U.S. Pat. Nos. 6,797,283; 6,764, 697; 6,635,268, and 6,648,083.

If desired, the components may be provided in a kit. The kit can additionally include instructions for using the kit. In some embodiments, the kit includes in one or more containers the NMDA receptor antagonist and, separately, in one or more containers, the fumarate agent. In other embodiments, the kit provides a combination with the NMDA receptor antagonist and the fumarate agent mixed in one or more containers. The kits include a therapeutically effective dose of an agent for treating seizure or pain-related conditions.

The NMDA receptor antagonist, the fumarate agent or both agents may be provided in a controlled, extended release form. In one example, at least 50%, 90%, 95%, 96%, 97%, 98%, 99%, or even in excess of 99% of the NMDA receptor antagonist is provided in an extended release dosage form. A release profile, i.e., the extent of release of the NMDA receptor antagonist or the fumarate agent over a desired time, may be conveniently determined for a given time by calculating the Cₘₐₓ/Cₘₑₐₙ for a desired time range to achieve a given acute or chronic steady state serum concentration profile. Thus, upon the administration to a subject (e.g., a mammal such as a human), the NMDA receptor antagonist has a Cmax/Cmean of approximately. 2.5, 2, 1.5, or 1.0 approximately 1, 1.5, 2 hours to at least 6, 8, 9, 12, 18, 21, 24 hours following such administration. If desired, the release of the NMDA receptor antagonist may be monophasic or multiphasic (e.g., biphasic). Moreover, the fumarate agent may be formulated as an extended release composition, having a Cₘₐₓ/Cₘₑₐₙₛ of approximately 2.5, 2, 1.5, or 1.0, approximately 1, 1.5, 2 hours to at least 6, 8, 9, 12, 18, 21, 24 hours following administration to a subject. One of ordinary skill in the art can prepare combinations with a desired release profile using the NMDA receptor antagonists and the fumarate agent and formulation methods known in the art or described below.

As shown in Tables 1 and 2, the pharmacokinetic properties of both of the drug classes vary from about 3 hours to more than 60 hours. Thus, one aspect of this invention is to select suitable formulations to achieve nearly constant concentration profiles over an extended period (preferably from 8 to 24 hours) thereby maintaining both components in a constant ratio and concentration for optimal therapeutic benefits for both acute and chronic administration. Preferred Cratio.var values are less than about 100%, 70%, 50%, 30%, 20%, 10%. Preferred Cratio.var values may be less than about 10%, 20%, 30%, 50%, 75%, or 90% of those for IR administration of the same active pharmaceutical ingredients over the first 4, 6, 8, 12 hours after administration.

Formulations that deliver this constant, measurable profile also allow one to achieve a monotonic ascent from an acute ratio to a desired chronic ratio for drugs with widely varying absorption rates and/or elimination half-lives. Compositions of this type and methods of treating patients with these compositions are embodiments of the invention. Numerous ways exist for achieving the desired release profiles, as described below.

Suitable methods for preparing combinations in which the first component, second component, or both components are provided in extended release-formulations include those described in U.S. Pat. No. 4,606,909 (hereby incorporated by reference). This reference describes a controlled release multiple unit formulation in which a multiplicity of individually coated or microencapsulated units are made available upon disintegration of the formulation (e.g., pill or tablet) in the stomach of the animal (see, for example, column 3, line 26 through column 5, line 10 and column 6, line 29 through column 9, line 16). Each of these individually coated or microencapsulated units contains cross-sectionally substantially homogenous cores containing particles of a sparingly soluble active substance, the cores being coated with a coating that is substantially resistant to gastric conditions but which is erodable under the conditions prevailing in the small intestine.

The combination may alternatively be formulated using the methods disclosed in U.S. Pat. No. 4,769,027, for example. Accordingly, extended release formulations involve prills of pharmaceutically acceptable material (e.g., sugar/starch, salts, and waxes) may be coated with a water permeable polymeric matrix containing an NMDA receptor antagonist and next overcoated with a water-permeable film containing dispersed within it a water soluble particulate pore forming material.

One or both components of the combination may additionally be prepared as described in U.S. Pat. No. 4,897,268, involving a biocompatible, biodegradable microcapsule delivery system. Thus, the NMDA receptor antagonist may be formulated as a composition containing a blend of free-flowing spherical particles obtained by individually microencapsulating quantities of memantine, for example, in different copolymer excipients which biodegrade at different rates, therefore releasing memantine into the circulation at a predetermined rates. A quantity of these particles may be of such a copolymer excipient that the core active ingredient is released quickly after administration, and thereby delivers the active ingredient for an initial period. A second quantity of the particles is of such type excipient that delivery of the encapsulated ingredient begins as the first quantity's delivery begins to decline. A third quantity of ingredient may be encapsulated with a still different excipient which results in delivery beginning as the delivery of the second quantity beings to decline. The rate of delivery may be altered, for example, by varying the lactide/glycolide ratio in a poly(D,L-lactide-co-glycolide) encapsulation. Other polymers that may be used include polyacetal polymers, polyorthoesters, polyesteramides, polycaprolactone and copolymers thereof, polycarbonates, polyhydroxybuterate and copolymers thereof, polymaleamides, copolyaxalates and polysaccharides.

Alternatively, the combination may be prepared as described in U.S. Pat. No. 5,395,626 features a multilayered controlled release pharmaceutical dosage form. The dosage form contains a plurality of coated particles wherein each has multiple layers about a core containing an NMDA receptor antagonist and/or the fumarate agent whereby the drug containing core and at least one other layer of drug active is overcoated with a controlled release barrier layer therefore providing at least two controlled releasing layers of a water soluble drug from the multilayered coated particle.

In some embodiments, the first component and second component of the combination described herein are provided within a single or separate pharmaceutical compositions. “Pharmaceutically or Pharmacologically Acceptable” includes molecular entities and compositions that do not produce an adverse, allergic or other untoward reaction when administered to an animal, or a human, as appropriate. “Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. “Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The preparation of pharmaceutical or pharmacological compositions are known to those of skill in the art in light of the present disclosure. General techniques for formulation and administration are found in “Remington: The Science and Practice of Pharmacy, Twentieth Edition,” Lippincott Williams & Wilkins, Philadelphia, Pa. Tablets, capsules, pills, powders, granules, dragées, gels, slurries, ointments, solutions suppositories, injections, inhalants and aerosols are examples of such formulations.

By way of example, extended release oral formulation can be prepared using additional methods known in the art. For example, a suitable extended release form of the either active pharmaceutical ingredient or both may be a matrix tablet composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm oil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups.

The coating composition may be plasticised according to the properties of the coating blend such as the glass transition temperature of the main component or mixture of components or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium.

The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

The pharmaceutical composition described herein may also include a carrier such as a solvent, dispersion media, coatings, antibacterial and antifungal agents, isotonic and absorption delaying agents. The use of such media and agents for pharmaceutically active substances is well known in the art. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, may also be used as a carrier.

Additional methods for making controlled release formulations are described in, e.g., U.S. Pat. Nos. 5,422,123, 5,601,845, 5,912,013, and 6,194,000, all of which are hereby incorporated by reference.

Preparation for delivery in a transdermal patch can be performed using methods also known in the art, including those described generally in, e.g., U.S. Pat. Nos. 5,186,938 and 6,183,770, 4,861,800, and 4,284,444. A patch is a particularly useful embodiment in this case owing to absorption problems with many fumarate agent. Patches can be made to control the release of skin-permeable active ingredients over a 12 hour, 24 hour, 3 day, and 7 day period. In one example, a 2-fold daily excess of an NMDA receptor antagonist is placed in a non-volatile fluid along with an fumarate agent. Given the amount of the agents employed herein, a preferred release will be from 12 to 72 hours.

Transdermal preparations of this form will contain from 1% to 50% active ingredients. The compositions of the invention are provided in the form of a viscous, non-volatile liquid. Preferably, both members of the combination will have a skin penetration rate of at least 10-9 mole/cm²/hour. At least 5% of the active material will flux through the skin within a 24 hour period. The penetration through skin of specific formulations may be measures by standard methods in the art (for example, Franz et al., J. Invest. Derm. 64:194-195 (1975)).

In some embodiments, for example, the composition may be delivered intranasally to the brain rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Preparation of a pharmaceutical composition for delivery in a subdermally implantable device can be performed using methods known in the art, such as those described in, e.g., U.S. Pat. Nos. 3,992,518; 5,660,848; and 5,756,115.

Indications Suitable for Treatment with the Combination

The combination of the NMDA receptor antagonist and fumarate can be used to treat any subject having or at risk of having an auto-immune disease, including a demyelinating condition. Demyelinating conditions include, for example, multiple sclerosis (MS); progressive multifocal leukoencephalopathy (PML); disseminated necrotizing leukoencephalopathy (DNL); acute disseminated encephalomyelitis; Schilder disease, central pontine myelinolysis (CPM); radiation necrosis; and Binswanger disease (SAE); Guillain-Barre Syndrome; leukodystrophy; acute disseminated encephalomyelitis (ADEM); acute transverse myelitis; acute viral encephalitis; adrenoleukodystrophy (ALD); adrenomyeloneuropathy; AIDS-vacuolar myelopathy; experimental autoimmune encephalomyelitis (EAE); experimental autoimmune neuritis (EAN); HTLV-associated myelopathy; Leber's hereditary optic atrophy; subacute sclerosing panencephalitis; and tropical spastic paraparesis. The combination may additionally be used to treat psoriasis.

The combination may additionally be used to treat a CNS-related condition. CNS-related conditions, such as epilepsy, seizure disorders, acute pain, chronic pain, chronic neuropathic pain may be treated using the combinations and methods described herein. Epileptic conditions include complex partial, simple partial, partials with secondary generalization, generalized—including absence, grand mal (tonic clonic), tonic, atonic, myoclonic, neonatal, and infantile spasms. Additional specific epilepsy syndromes are juvenile myoclonic epilepsy, Lennox-Gastaut, mesial temporal lobe epilepsy, nocturnal frontal lobe epilepsy, progressive epilepsy with mental retardation, and progressive myoclonic epilepsy. The combinations of the invention are also useful for the treatment and prevention of other disorders including headaches (e.g., migraine, tension, and cluster), cerebrovascular disease, motor neuron diseases (e.g., ALS, Spinal motor atrophies, Tay-Sach's, Sandoff disease, familial spastic paraplegia), dementias (e.g., Alzheimer's disease, Parkinson's disease, Picks disease, fronto-temporal dementia, vascular dementia, normal pressure hydrocephalus, HD, and MCI), neurodegenerative diseases (e.g., familial Alzheimer's disease, prion-related diseases, cerebellar ataxia, Friedrich's ataxia, SCA, Wilson's disease, RP, ALS, Adrenoleukodystrophy, Menke's Sx, cerebral autosomal dominant arteriopathy with subcortical infarcts (CADASIL); spinal muscular atrophy, familial ALS, muscular dystrophies, Charcot Marie Tooth diseases, neurofibromatosis, von-Hippel Lindau, Frangile X, spastic paraplesia, Tuberous sclerosis, and Wardenburg syndrome), strokes (e.g, thrombotic, embolic, thromboembolic, hemmorhagic, venoconstrictive, and venous), movement disorders (e.g., PD, dystonias, benign essential tremor, tardive dystonia, tardive dyskinesia, and Tourette's syndrome), ataxic syndromes, disorders of the sympathetic nervous system (e.g., Shy Drager, Olivopontoicerebellar degeneration, striatonigral degenration, PD, HD, Gullian Barre, causalgia, complex regional pain syndrome types I and II, diabetic neuropathy, and alcoholic neuropathy), Cranial nerve disorders (e.g., Trigeminal neuropathy, trigeminal neuralgia, Menier's syndrome, glossopharangela neuralgia, dysphagia, dysphonia, and cranial nerve palsies), myelopethies, traumatic brain and spinal cord injury, radiation brian injury, Multiple sclerosis, Post-menengitis syndrome, prion diseases, myelities, radiculitis, neuropathies (e.g., Guillian-Barre, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy), pain syndromes (e.g., acute, chronic, neuropathic, nociceptive, central, and inflammatory), axonic brain damage, encephalopathies, chronic fatigue syndrome, psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (eg, crack, cocaine, speed, meth), opioids, and nicotine). Any of these conditions may be treated using the methods and compositions described herein.

Treatment of a subject with the combination may be monitored using methods known in the art. The efficacy of treatment using the combination is preferably evaluated by examining the subject's symptoms in a quantitative way, e.g., by noting a decrease in the frequency of relapses, or an increase in the time for sustained worsening of symptoms. In a successful treatment, the subject's status will have improved (i.e., frequency of relapses will have decreased, or the time to sustained progression will have increased).

Additional embodiments are within the claims.

What is claimed is:

1. A pharmaceutical composition comprising an NMDA receptor antagonist and a fumarate agent.

2. The pharmaceutical composition of claim 1, wherein the fumarate agent is a fumaric acid ester.

3. The pharmaceutical composition of claim 1, wherein the fumarate agent is dialkyl fumarate.

4. The pharmaceutical composition of claim 1, wherein at least one of the NMDA receptor antagonist or fumarate, agent is provided in an extended release dosage form.

5. The pharmaceutical composition of claim 1, wherein said fumarate agent is dimethyl fumarate.

6. The pharmaceutical composition of claim 1, wherein said NMDA receptor antagonist is memantine.

7. The pharmaceutical composition of claim 1, wherein said NMDA receptor antagonist is memantine and the fumarate agent is dimethyl fumarate.

8. A method of treating a demyelinating condition comprising administering to a subject having a demyelinating condition an NMDA receptor antagonist and a fumarate agent.

9. The method of claim 8, wherein said demyelinating condition is multiple sclerosis.

10. The method of claim 8, wherein said NMDA receptor antagonist is memantine.

11. The method of claim 8, wherein said fumarate agent is a fumarate ester.

12. The method of claim 8, wherein said fumarate agent is a dialkyl fumarate.

13. The method of claim 8, wherein said fumarate agent is dimethyl fumarate.

14. The method of claim 8, further comprising administering to said subject an additional multiple sclerosis agent selected from the group consisting of β-interferons, glatiramer acetate, natalizumab, mitoxanthrone, and daclizumab.

15. A kit for treating a patient having multiple sclerosis, comprising a therapeutically effective dose of a fumarate agent and a NMDA receptor antagonist, either in the same or separate packaging, and instructions for its use.

16. The kit of claim 16, further including an additional agent selected from the group consisting of β-interferons, glatiramer acetate, natalizumab, mitoxanthrone, and daclizumab.

17. The kit of claim 16, wherein said NMDA receptor antagonist is selected from the group consisting of memantine, rimantadine, and amantadine.

18. The kit of claim 16, wherein said fumarate agent is dimethyl fumarate.
